(19) United States
(12) Patent Application Publication
(10) Pub. No.: US 2026/0192044 A1
(43) Pub. Date: Jul. 9, 2026

(54) DEDICATED CONSUMABLE SETS FOR SYRINGE INFUSION PUMPS

(71) Applicant: ICU Medical, Inc., San Clemente, CA (US)

(72) Inventors: James Jacobson, Lindenhurst, IL (US); Robert Butterfield, Poway, CA (US); Robert Hlinsky, Glenview, IL (US)

(21) Appl. No.: 19/132,460

(22) PCT Filed: Dec. 8, 2023
(86) PCT No.: PCT/US2023/083036

§ 371 Date: May 23, 2025

Related U.S. Application Data
(60) Provisional application No. 63/431,421, filed on Dec. 9, 2022.
(60) Provisional application No. 63/435,996, filed on Dec. 29, 2022.

(51) Int. Cl. A61M 5/145; A61M 5/168

(52) U.S. Cl. CPC A61M 5/1456; A61M 5/16813; A61M 5/16854; A61M 2205/3334; A61M 2205/60

(57) ABSTRACT

Dedicated consumable sets with pressure sensors and valves, for use with syringe infusion pumps, are disclosed. A dedicated consumable set for use with a syringe pump may include a housing, a valve positioned between distal and proximal ends of the dedicated consumable set and configured to block or regulate the flow of infusate through tubing from a syringe to a patient, and a pressure sensor for measuring pressure inside the dedicated consumable set, and optionally a flow sensor for measuring flow, the pressure sensor configured to transmit pressure measurements above a predetermined level to the valve. The valve can be further configured to automatically actuate between an open position and a closed position, or to proportionally adjust restriction and modulate contained volume, in response to an external event, which may be receiving a pressure measurement above a predetermined level or detecting the removal of the syringe from the syringe pump.

### CROSS REFERENCE TO RELATED APPLICATIONS

This application claims the benefit of U.S. Provisional Application No. 63/431,421, filed Dec. 9, 2022, and U.S. Provisional Application No. 63/435,996, filed Dec. 29, 2022, the disclosures of which are hereby incorporated by reference.

### TECHNICAL FIELD

The present disclosure generally pertains to dedicated consumable sets offering advanced functionality when used with infusion pumps. The present disclosure particularly pertains to dedicated consumable sets with pressure sensors, flow rate sensors, and/or valves for use with infusion pumps.

### BACKGROUND

Infusion pumps are extremely useful medical devices for providing prescribed fluids, drugs, and other therapies (collectively, “infusates”) to patients in controlled amounts. For example, medications such as antibiotics, chemotherapy drugs, vasoactives, insulin, blood products, and pain relievers are commonly delivered to patients via infusion pumps, as are nutrients and other supplements. Infusion pumps have been used in hospitals, nursing homes, and in other short-term and long-term medical facilities, as well as for in-home care. Infusion pumps can be particularly useful for the delivery of medical therapies requiring an extended period of time for their administration. There are many types of infusion pumps, including large volume, patient-controlled analgesia (PCA), elastomeric, syringe (syringe driver), enteral, and insulin pumps. Infusion pumps are typically useful in various routes of medication delivery, including intravenously, intra-arterially, subcutaneously, intraperitoneally, intraosseous, intraportal, in close proximity to nerves, and into an intraoperative site, epidural space or subarachnoid space.

Syringe pumps have several desirable characteristics and are generally perceived as the most precise and accurate acute care infusion pumps available. Syringe pumps may support lower flow rates than large volume pumps or ambulatory pumps, sometimes as low as 0.01 milliliters/hour (mL/hr) with appropriately-sized small syringes. Unlike large volume and ambulatory pumps that may often utilize proprietary or dedicated consumables, syringe pumps typically accommodate wide ranges of commonly used or “off-the-shelf” syringe brands and sizes that are typically coupled with non-proprietary extension sets for delivering infusates to patients.

Some syringe pumps can suffer from performance limitations, particularly at low flow rates (below 5 mL/hr and particularly below 0.1 mL/hr), including but not limited to long times to reach target flow rates, inconsistent flow profiles during infusate delivery, long times to detect an occlusion, and risk of inadvertently delivering a bolus or allowing retrograde flow due to varying external pressure. Further dynamic flow inaccuracies may arise from changes in backpressure due to multiple pumps being added to, or removed from, the same infusion line.

Off the shelf or non-dedicated tubing consumables have traditionally supported only fluid routing between a syringe pump and a patient access device (such as a catheter) for delivery of fluid. While the traditional use of simple off-the-shelf or non-dedicated tubing sets provides clinicians with flexibility in selecting different tubing set lengths, materials, priming volumes, and compliance characteristics from among numerous vendors, there is a need for more sophisticated consumable tubing sets to integrate more fully with pump operation. Dedicated tubing sets will provide improved operation including accelerated start-up time and occlusion detection time, and the ability to address other known operating issues with syringe pumps.

Upon initiation of a programmed infusion via a syringe pump, there can be a substantial delay before the programmed rate is achieved, particularly at low rates and with large volume syringes. This is due to mechanical slack in the pump system that must be absorbed by the syringe pump mechanism before the mechanism starts to effectively drive the syringe plunger into the syringe barrel. Once the pump mechanism has engaged with the syringe plunger and started forcing the syringe plunger into the syringe barrel, it takes further time before the system is operating at the desired programmed rate as compliance of the pump, syringe, and tubing set absorbs incremental fluid volume until a steady state flow rate is achieved. At low and very low rates, and/or with large volume syringes, it can typically take hours for the system to reach a target flow rate. Aspects of this disclosure greatly accelerate the start-up time of syringe pumps, particularly at low and very low rates.

During operation of a syringe pump delivering infusates, an occlusion might occur when the fluid line is clamped, blocked or otherwise impeded, as when for example the infusion line tubing is kinked or the catheter becomes blocked due to formation of thrombotic or non-thrombotic blockage. If the occlusion is not noticed by a care provider or detected by the syringe pump, the patient likely would not receive the prescribed medication leading to potentially serious consequences. At relatively low flow rates, the amount of time to detect an occlusion may be unacceptably long as intended and commanded forward progression of the plunger does not deliver fluid to the patient, but rather introduces fluid which is stored as incremental volume within the compliances of the pump, the syringe, or the set.

Some current syringe pumps include a force sensor in the plunger driver head which indirectly senses the fluid pressure by measurement of syringe plunger force. When the force or rate of change of force detected by that sensor exceeds predetermined thresholds, a processor monitoring that signal generates an indication that an occlusion has possibly occurred or is possibly occurring presently and may pause movement of the driver head. Since syringe pumps are typically capable of accommodating a wide range of syringe diameters or sizes (e.g., 1 mL through 100 mL capacities) and exhibit a wide range of stopper friction forces, the driver head and force sensor may experience varying occlusion forces depending upon the syringe being used leading to varying accuracy and responsiveness overall in the pump's occlusion sensing system. Because syringe pumps typically need to accommodate a wide range of syringe sizes, the accuracy of detecting an occlusion at low flow rates and/or with smaller syringes may be decreased. Aspects of the current disclosure greatly reduce the time required for the pump system to recognize an occlusion, particularly at low rates.

While valuable improvements to syringe pump constructions, configurations, and operations have been and continue to be made, there remains a need for improvements to consumable tubing sets themselves and associated components for their uses in conjunction with syringe pumps. The present disclosure addresses these concerns.

### SUMMARY

Embodiments described or otherwise contemplated herein substantially provide the advantages of improving ease of use, operation, accuracy, and patient safety in the delivery of infusates, among other advantages.

In embodiments, a dedicated consumable set for use with a syringe pump may comprise a housing, a valve positioned between distal and proximal ends of the dedicated consumable set and configured to block or regulate the flow of infusate through tubing from a syringe to a patient, and a direct fluid pressure sensor for measuring pressure inside the dedicated consumable set, and optionally a flow sensor for measuring fluid flow from the syringe. The pressure sensor is configured to transmit pressure measurements to a processor which uses an algorithm to control the valve and in some implementations the pump motor also or alternatively, wherein the valve is further configured to automatically actuate between an open position and a closed position, or to proportionally adjust restriction, in response to an external pressure event.

In embodiments, the external event is receiving a pressure measurement above the predetermined level. In embodiments, the external event is the removal of the syringe from the syringe pump.

The present disclosure provides the following improvements and advantages over prior approaches:
an ability to rapidly achieve a programmed infusion rate;
an ability to rapidly recognize and alarm a line occlusion;
an ability to reduce post occlusion bolus volume;
an ability to rapidly recognize changes in flow delivery, potentially due to dynamics of other syringe pumps connected to the same patient delivery line;
an ability to minimize unintended boluses or retrograde flow when the syringe/consumable set is removed from the syringe pump;
an ability to minimize unintended boluses or retrograde flow when the consumable set is disconnected from the syringe;
a potential to sense line disconnections if a syringe extension line is accidentally disconnected from a patient access device, or a patient access device connected to the infusion set is accidentally removed from the patient; and
a potential to minimize unintended boluses due to head height changes while the syringe pump is infusing.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a perspective view of an example of a syringe pump for use with embodiments of the disclosure.

FIG. 2 is a schematic view of a syringe and dedicated set assembly, according to an embodiment of the disclosure.

FIG. 3 is a schematic view of a syringe and dedicated set assembly, according to an embodiment of the disclosure.

FIG. 4 is a schematic view of a method of operating a syringe and dedicated set assembly, according to an embodiment of the disclosure.

FIG. 5 is a schematic view of a method of operating a syringe and dedicated set assembly, according to an embodiment of the disclosure.

FIG. 6 is a schematic view of a method of operating a syringe and dedicated set assembly, according to an embodiment of the disclosure.

FIG. 7 is a schematic view of a method of operating a syringe and dedicated set assembly, according to an embodiment of the disclosure.

FIG. 8 is a perspective view of a dedicated consumable set, according to an embodiment of the disclosure.

FIG. 9 is a perspective view of a syringe pump system, according to an embodiment of the disclosure.

While various embodiments are amenable to various modifications and alternative forms, specifics thereof have been shown by way of example in the drawings and will be described in detail. It should be understood, however, that the intention is not to limit the claimed subject matter to the particular embodiments described. On the contrary, the intention is to cover all modifications, equivalents, and alternatives falling within the spirit and scope of the subject matter as defined by the claims.

### DETAILED DESCRIPTION OF THE DRAWINGS

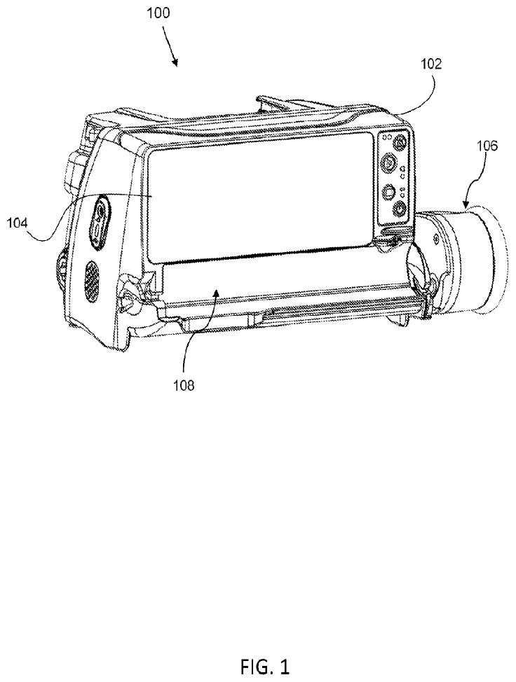

Referring now to FIG. 1, an example of a syringe pump 100 is depicted for use with embodiments of the present disclosure. Syringe pump 100 can include a housing 102, user interface 104, a syringe drive assembly 106, and a syringe receptacle 108.

The syringe drive assembly 106 can be employed for controlling delivery of a prescribed amount or dose of an infusate from a syringe that has been installed in the pump 100 (not illustrated in FIG. 1) to a patient by mechanically advancing a plunger in the syringe barrel to deliver the infusate at a controlled rate through an infusion line fluidly connected to the syringe. In an example, a motor within pump 100 rotates a lead screw which, in turn, causes a plunger driver head assembly of the syringe drive assembly 106 to move in a direction generally along a longitudinal axis of the syringe receptacle 108. This movement then pushes the plunger within a barrel of the syringe located within the receptacle 108, where the barrel is held substantially in place. Moving the syringe plunger forward acts to displace a volume of infusate in the syringe outwardly from the syringe, into the infusion line, and ultimately to the patient.

In the example pump 100 of FIG. 1, the syringe receptacle 108 provides a cavity extending across the front of the syringe pump 100 such that a syringe installed therein is readily and sustainably visible. The syringe receptacle 108 is shaped and sized to accept installation of syringes of various sizes and brands therein for delivery of infusates.

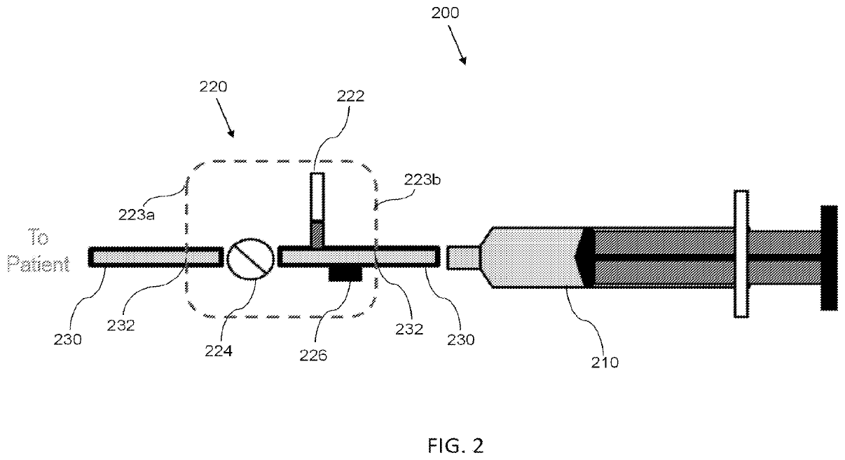

Referring now to FIG. 2, in an embodiment, a syringe and dedicated tubing set assembly 200 for use with a syringe pump 100 (not shown) includes a syringe 210 connected to a dedicated consumable set 220. The dedicated consumable set 220 can include a pressure sensor 222, a valve 224, a radio-frequency identification (RFID) tag 226 (optional), tubing 230, and tubing connectors 232.

Pressure sensor 222 may be positioned between valve 224 and an inlet of dedicated consumable set 220 where syringe 210 is connected. Pressure sensor 222 is configured to monitor pressure in-line (or nearly in-line) which can more accurately measure lower pressures compared to prior approaches relying on a force sensor connected with the syringe drive assembly 106 of syringe pump 100. Pressure sensor 222 can be electrically, mechanically, or otherwise interfaced with syringe pump 100, though other embodiments where pressure sensing can be accomplished with reusable pressure sensing across a flexible membrane are contemplated. Pressure sensor 222 may comprise any pressure measurement device that is known in the art and is adaptable for use with syringe and dedicated set assembly 200 and syringe pump 100.

As depicted in FIG. 2 for example, the arrangement of pressure sensor 222 can provide insight into start-up operations of syringe pump 100 as well as additional control improvements during operation. For example, during start-up operations, the in-line pressure may provide an approach for determining indirectly when a plunger in the syringe 210 has commenced forward movement. In operation, when the syringe pump 100 is started, the valve 224 is initially closed. If pressure does not increase, the speed of the syringe pump 100 can be increased temporarily until pressure rises to a given level or the pressure rate of change reaches a given level. At this point in operation, the valve 224 would be opened, either completely or partially, to permit infusion in the line to occur. Additional control improvements may include occlusion detection, stick-slip detection, and disconnect detection, as discussed herein.

In an embodiment, valve 224 may be positioned downstream of pressure sensor 222, as depicted in FIG. 2 for example. Valve 224 can be used to selectively isolate downstream tubing 230 from syringe 210 such that infusate delivery through tubing 230 is blocked. In embodiments, valve 224 can be configured to alternate between an open position and a closed position in response to an external event or a signal received from syringe pump 100. Valve 224 may comprise any device used to control fluid passage that is known in the art and is adaptable for use with syringe and dedicated set assembly 200. Examples of suitable valves 224 include, but are not limited to, a traditional stopcock valve or a valve that can be electrically driven from syringe pump 100, and also completely disposable (e.g., a micro-solenoid valve, or a proportioning valve that may be party opened or closed and may be configured to add or subtract very small volumes to the fluid flow path to assist in mitigating unintended flow transients).

RFID tag 226 can be configured as active or passive. RFID tag 226 may include identifying information pertaining to the set, such as tubing length, tubing diameter, priming volume, date of manufacture, type of pressure sensor 222, type of valve 224, or other information as desired (e.g., enteral, intravenous, and subcutaneous epidural delivery route identification). RFID tag 226 may be communicable with syringe pump 100 or other devices, such as an RFID tag reader operable by a clinician.

Tubing 230 is configured to attach to a tubing connector 232 positioned on each end of dedicated consumable set 220 to create a pathway for the flow of infusate from syringe 210 through dedicated consumable set 220 and to a patient. Additional sections of tubing 230 may also be provided as desired, for example between pressure sensor 222 and valve 224. In embodiments, tubing 230 may be provided between an outlet of syringe 210 and an inlet of dedicated consumable set 220. Tubing 230 may also be provided between an outlet of dedicated consumable set 220 and a patient access device, such as a catheter. Tubing 230 and tubing connectors 232 may be manufactured from a medical-grade plastic including, but not limited to, silicone, polyethylene, polyurethane, or polyvinyl chloride.

In embodiments, dedicated consumable set 220 may further include particulate or air elimination filters and light-resistant tubing or housing (both not depicted). Filters would be added to eliminate particulate, bacteria, or virus accumulation in dedicated consumable set 220. Light-resistant components of dedicated consumable set would help eliminate particulates, bacteria, or viruses in certain IV fluids that are susceptible to degradation from room light.

In embodiments, a simple occlusion detection algorithm with long data averaging windows and a simple alarm definition may be used with syringe and dedicated set assembly 200, for example monitoring line pressure through pressure sensor 222. Alternatively, a more sophisticated algorithm for processing data may be used, including, but not limited to, monitoring changes in line pressure similar to force monitoring in FlowSentry™ as used with the MEDFUSION® 3500 and 4000 systems from Smiths Medical, Inc.

Alternatively, the in-line pressure sensor 222 may be useful for occlusion detection by measurement of both passive and active (deliberately induced) sources of pressure. For example, when a hard occlusion occurs, the normal patient pressures are no longer present providing a predictive indicator that an occlusion may have occurred. Another example is use of a proportional valve to introduce small volume perturbations in the fluid. When an occlusion is present, the pressure response will be distinguishable from normal conditions providing a further predictive indicator. The use of an in-line pressure sensor 222 is superior to a conventional force sensor located within the plunger because it permits setting of lower pressure limits which in turn shorten occlusion detection time.

In an example “fast start”, “rapid start-up”, or “accelerated start-up” implementation, flow may commence at a rate substantially greater than the programmed rate and then decelerate over time until the programmed rate is reached. This allows the programmed rate to be achieved quickly and without risk of overshooting to an unacceptable rate. Pressure sensor 222 and optionally a flow sensor (not depicted) in the line may allow such implementations to operate more efficiently and accurately based on measurement of plunger forces.

In embodiments, a simple accelerated start-up algorithm utilizing an increased initial mechanical drive speed while monitoring line pressure to recognize removal of mechanical slack from the system and incremental absorption of system compliance could be used to accelerate start-up, with reduction of the mechanism to programmed rate after a threshold pressure profile is attained. Alternatively, a more sophisticated algorithm of an accelerate mechanism drive rate followed by discrete deceleration steps could be applied to accelerate the start-up of syringe pump 100. For example, algorithms disclosed in U.S. Pat. No. 11,179,515, incorporated herein in its entirety, could be used.

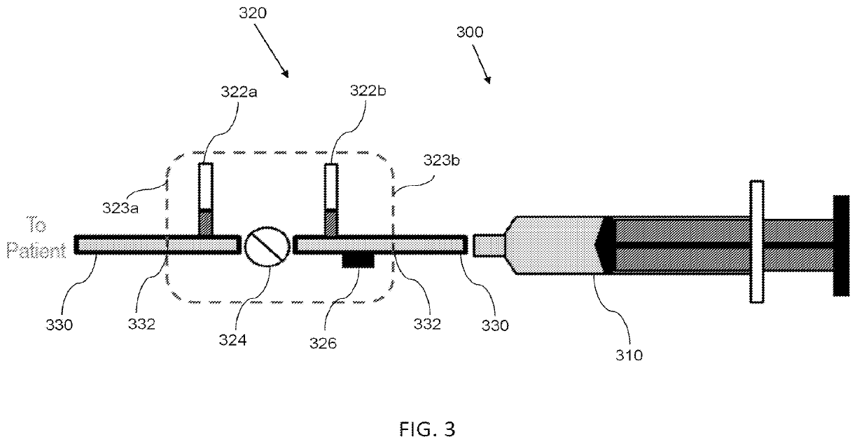

Referring now to FIG. 3, in an embodiment, a syringe and dedicated set assembly 300 for use with a syringe pump 100 (not shown) includes a syringe 310 connected to a dedicated consumable set 320, the dedicated consumable set 320 including a first pressure sensor 322a, a second pressure sensor 322b, a valve 324, an RFID tag 326, tubing 330, and tubing connectors 332. The embodiments of assembly 300 have many similarities to assembly 200, and for simplicity the description of common components is not repeated in the following, and like numerals may designate like parts throughout that are corresponding or analogous.

Dedicated consumable set 320 includes pressure sensors 322a, b positioned near distal and proximal ends 323a,b of dedicated consumable set 320, respectively, which offers additional control improvements for syringe and dedicated set assembly 300 over assembly 200 outlined in FIG. 2. For example, it could be advantageous to monitor downstream pressure via pressure sensor 322a to advise a target upstream pressure sensor 322b prior to opening valve 324 in order to accelerate start-up. This also allows for management of bolus due to head height changes and flow reflux due to connecting to an existing running infusion on another pump.

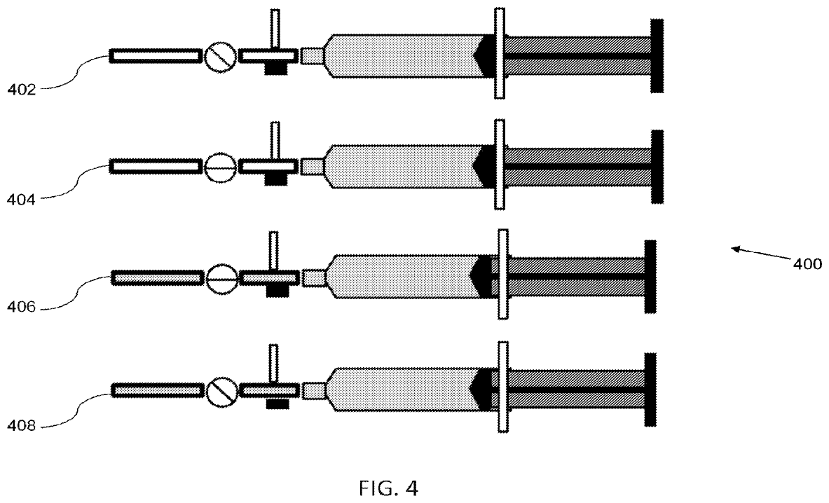

Referring now to FIG. 4, a method 400 of manually priming syringe and dedicated set assembly 200 is depicted, according to an embodiment. For simplicity in FIG. 4, some reference numerals have been omitted that correspond to stated components that have been previously depicted and described. At 402, dedicated consumable set 220 (not explicitly depicted in FIG. 4) having a closed valve 224 can be coupled to a syringe 210 filled with infusate to form syringe and dedicated set assembly 200. At 404, valve 224 is manually opened by a user of syringe and dedicated set assembly 200 which creates a path for infusate to flow through dedicated consumable set 220. At 406, the user transfers infusate from syringe 210 through tubing 230 to prime the line. At 408, the valve 224 is manually closed by the user to block further infusate flow from the syringe 210. In embodiments, method 400 may also be used with syringe and dedicated set assembly 300.

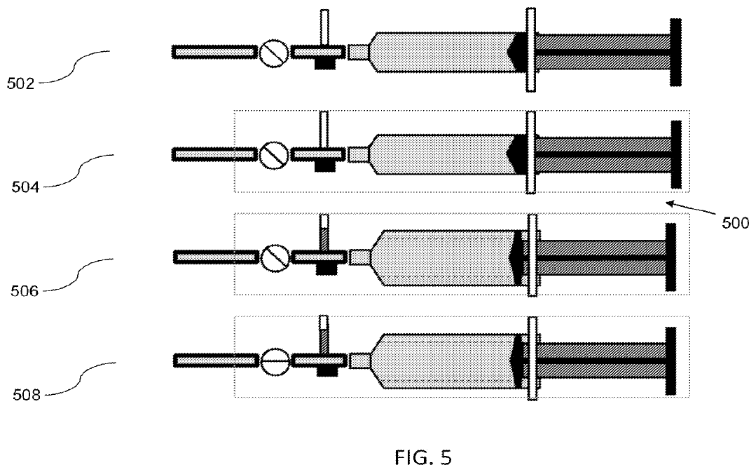

Referring now to FIG. 5, a method 500 of operating a syringe and dedicated set assembly 200 within a syringe pump 100 to accelerate start-up is depicted, according to an embodiment. For simplicity in FIG. 5, some reference numerals have been omitted that correspond to stated components that have been previously depicted and described. At 502, the syringe and dedicated set assembly 200 is primed by a user and valve 224 is manually closed. At 504, the syringe and dedicated set assembly 200 is loaded into a syringe pump 100 and connected to a patient. In embodiments, syringe and dedicated set assembly 200 can only be loaded into syringe pump 100 when valve 224 is closed. In embodiments, the cavity in the pump which receives a handle of valve 224 may act as a key permitting the dedicated consumable set 220 (not explicitly depicted in FIG. 5) to be loaded only with the handle positioned in a closed position. At 506, the syringe drive assembly 106 of syringe pump 100 is used to mechanically advance a plunger in syringe 210 to deliver infusate at a controlled rate while absorbing mechanical slack and compliance. At 508, valve 224 is opened which creates a pathway for infusate to flow from syringe 210 to the patient. In embodiments, method 500 may be used with syringe and dedicated set assembly 300.

Method 500 absorbs mechanical slack and compliance with both syringe and dedicated set assembly 200 and syringe pump 100, and also pressurizes syringe 210. Method 500 advantageously addresses the typically long periods of time for syringe pumps to reach accurate flow rates after start-up by allowing the pump mechanism to safely run at a higher than programmed rate while digesting mechanical slack and accommodating system compliance, thereby reducing the start-up time.

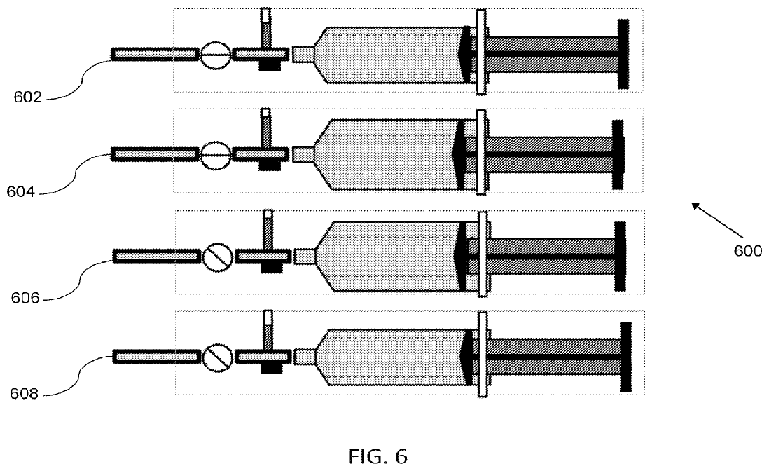

Referring now to FIG. 6, a method 600 of operating syringe and dedicated set assembly 200 to reduce post-occlusion bolus is depicted, according to an embodiment. For simplicity in FIG. 6, some reference numerals have been omitted that correspond to stated components that have been previously depicted and described. At 602, syringe pump 100 with syringe and dedicated set assembly 200 is pressurized to begin normal operation in a steady state delivery mode. At 604, a pressure increase is sensed by pressure sensor 222, signaling an occlusion. Typically, prior to alarming that an occlusion is present, a pump mechanism continues to drive the syringe plunger into the barrel, with the incremental fluid volume stored within compliance in the syringe and tubing set and therefore available as a post-occlusion bolus upon resolution of the occlusion. In the present disclosure, the presence of in-line pressure sensor 222 allows a lower pressure setting to reliably signal an occlusion which reduces the amount of time the syringe pump 100 continues to introduce excess fluid volume under pressure in the system.

At 606, detection of the occlusion is communicated by pressure sensor 222 to a valve 224 (either directly or through syringe pump 100) which subsequently causes valve 224 to close to restrict further infusate flow from syringe 210. The benefit of this approach compared to currently practiced post-occlusion bolus reduction is that since most of the bolus is stored in the compliance of the syringe 210 and is released to the patient if the occlusion is removed (by caregiver or unintended tubing movement), the bolus will be prevented from reaching the patient by the valve 224. Using the pressure sensor 222 output, the stored pressurized volume can be eliminated by reversing the syringe pump 100 until the pressure is suitably reduced.

At 608, syringe pump 100 reverses operating direction to decrease the force applied to syringe 210 which reduces the risk of inadvertently delivering a bolus to the patient. Finally, after removing the occlusion, syringe pump 100 resets to an equilibrium state and resumes normal operation with valve 224 opened. In embodiments, method 600 may be used with syringe and dedicated set assembly 300.

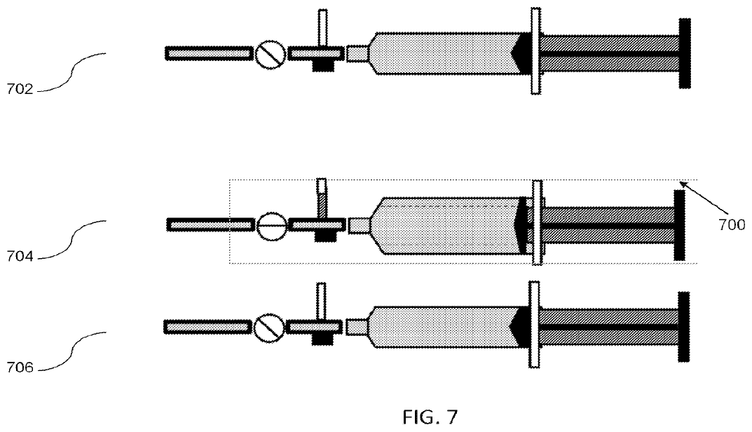

Referring now to FIG. 7, a method 700 is depicted for operating a syringe and dedicated set assembly 200 to minimize bolus and retrograde flow when a syringe 210 is removed from a syringe pump 100, according to an embodiment. For simplicity in FIG. 7, some reference numerals have been omitted that correspond to stated components that have been previously depicted and described. At 702, a syringe and dedicated set assembly 200 is loaded into a syringe pump 100. Syringe and dedicated set assembly 200 can only be loaded into syringe pump 100 when valve 224 is in the closed position. At 704, syringe pump 100 starts up and begins operation in a steady state delivery mode, with valve 224 in the open position. If syringe 210 is removed from syringe pump 100 either inadvertently or for a syringe swap, at 706, valve 224 is configured to automatically close. This advantageously helps reduce unintended boluses or retrograde flow in syringe and dedicated set assembly 200. In embodiments, method 700 may be used with syringe and dedicated set assembly 300.

Embodiments of the disclosure provide solutions to the issue of siphoning infusate outside of a syringe pump. Traditionally, a syringe and set outside the syringe pump is subject to gravity and other pressure-induced flows, which can result in siphoning of the medication from a syringe to the patient, or retrograde flow from the patient to the syringe. Through this disclosure, the normally closed valve 224 in the dedicated consumable set 220 introduces a layer of safety to keep the volume of fluid in the syringe 210 and dedicated consumable set 220 constant. In embodiments, valve 224 is configured to respond to other external events in addition to an increase in pressure communicated by pressure sensor 222 or removal of syringe 210 from syringe pump 100.

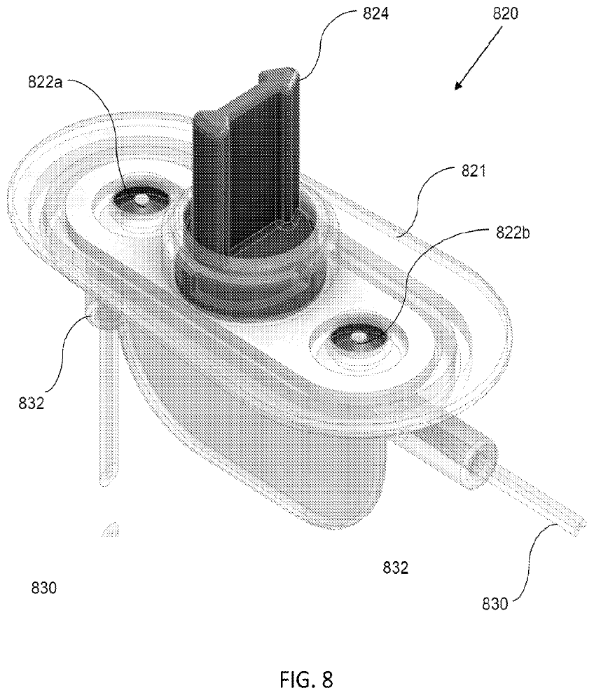

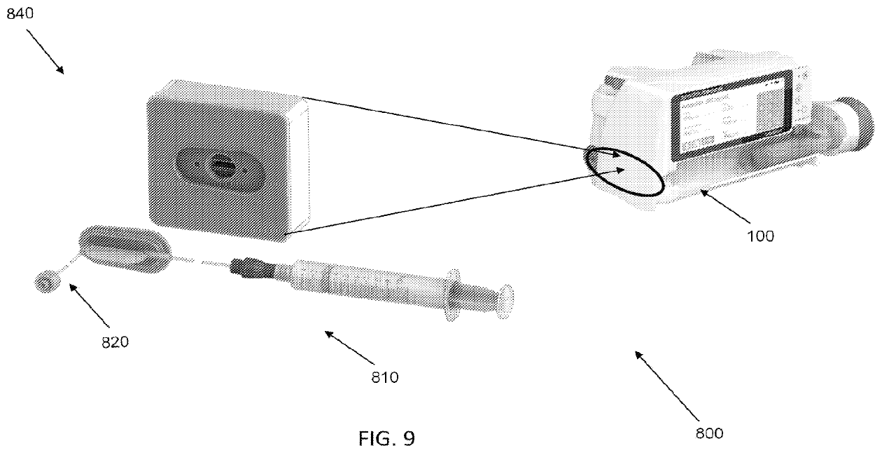

Referring now to FIG. 8 in an embodiment, a consumable set 820 includes a housing 821, a first pressure sensor communicating membrane 822a, a second pressure sensor communicating membrane 822b, a valve 824, tubing 830, and tubing connectors 832. The embodiments of consumable set 820 have many similarities to dedicated consumable set 320, and for simplicity the description of common components is not repeated in the following, and like numerals may designate like parts throughout that are corresponding or analogous. Of note, rather than introducing pressure sensors into consumable set 820, membranes 822a,b on cassette 820 communicate upstream and downstream pressures to reusable pressure sensors included in a hardware module (840 in FIG. 9), which provides an advantage of a purely mechanical interface between consumable set 820 and hardware module 840.

Referring now to FIG. 9, in an embodiment, a syringe pump system 800 includes a syringe pump 100, a syringe 810, a consumable set 820, and a hardware module 840. The embodiments of syringe 810 have many similarities to syringe 210 and for simplicity the description of common components is not repeated in the following, and like numerals may designate like parts throughout that are corresponding or analogous.

As indicated by FIG. 9, syringe 810 connects to consumable set 820 which can then be removably coupled to hardware module 840. Hardware module 840 may then be removably coupled to a side of syringe pump 100 for ease of access and consolidation of each component of syringe pump system 800 in a single location, thereby increasing efficiency of a user of syringe pump system 800. Alternatively, rather than a standalone hardware module 840, the functionality embedded in the hardware module 840 could be directly integrated into the side of the syringe pump 100.

Various embodiments of systems, devices, and methods have been described herein. These embodiments are given only by way of example and are not intended to limit the scope of the claimed subject matter. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed subject matter.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A dedicated consumable set for use with a syringe pump, comprising:
a housing;
a valve positioned between inlet and outlet ends of the dedicated consumable set and configured to block or regulate the flow of infusate through the set from a syringe coupled to the inlet to the outlet; and
a pressure sensor for measuring pressure inside the dedicated consumable set, the pressure sensor configured to transmit pressure measurements above a predetermined level to the valve,
wherein the valve is further configured to automatically actuate between an open position and a closed position, or to proportionally adjust restriction, in response to an external event.

2. The dedicated consumable set of claim 1, wherein the external event is receiving a pressure measurement above the predetermined level.

3. The dedicated consumable set of claim 1, wherein the external event is the removal of the syringe from the syringe pump.

4. The dedicated consumable set of claim 1, wherein the valve is arranged between the pressure sensor and the outlet.

5. The dedicated consumable set of claim 1, wherein the pressure sensor is configured to measure pressure in-line of the set.

6. The dedicated consumable set of claim 1, further comprising a second pressure sensor arranged between the valve and the outlet.

7. The dedicated consumable set of claim 1, further comprising an RFID tag including identifying information about the set.

8. A method of operating an infusion pump to reduce post-occlusion bolus, the infusion pump configured to operate with a syringe and an infusion set including a housing, a valve configured to block or regulate a flow of liquid through the set, and a pressure sensor for measuring pressure inside the infusion set, the method comprising:
pressurizing the syringe and the infusion set to begin operation in a steady state delivery mode;
sensing a pressure increase by the pressure sensor indicative of an occlusion;
closing the valve in response to the occlusion;
reversing operation direction of the infusion pump to decrease the force applied to the syringe to reduce any accumulated bolus; and
opening the valve after clearing the occlusion such that operation of the infusion pump in steady state delivery mode can resume.

9. The method of claim 8, wherein the valve is arranged between the pressure sensor and an outlet of the infusion set.

10. The method of claim 8, wherein the pressure sensor is configured to measure pressure in-line of the set.

11. The method of claim 8, wherein the infusion set includes an RFID tag having identifying information about the set, the method further comprising modifying an operating parameter of the infusion pump based on information from the RFID tag of the infusion set.

12. The method of claim 8, further comprising automatically closing the valve in response to detecting removal of the syringe.

13. A system for delivering infusate from a syringe, the system comprising:
a syringe pump including a housing, a syringe drive assembly, and a syringe receptacle comprising a cavity extending across a front of the housing; and
an infusion set including a valve operable to block or regulate a flow of infusate through the set, and a pressure sensor for measuring pressure inside the infusion set,
wherein the valve is configured to automatically actuate between an open position and a closed position, or to proportionally adjust restriction, in response to an external event.

14. The system of claim 13, wherein the external event is receiving a pressure measurement from the pressure sensor that is above a predetermined level.

15. The system of claim 13, wherein the external event is removal of the syringe from the syringe pump.

16. The system of claim 13, wherein the valve is arranged between the pressure sensor and the outlet.

17. The system of claim 13, wherein the valve is positioned between inlet and outlet ends of the set, and wherein the syringe is coupleable to an inlet of the set.

18. The system of claim 13, wherein the pressure sensor is configured to measure pressure in-line of the infusion set.

19. The system of claim 13, wherein the infusion set further includes an RFID tag including identifying information about the infusion set.

20. The system of claim 19, wherein an operating parameter of the syringe pump is modified based on information from the RFID tag of the infusion set.
